(19) United States
(12) Patent Application Publication
(10) Pub. No.: US 2004/0181186 A1
(43) Pub. Date: Sep. 16, 2004

(54) Medical device

(75) Inventors: Barry N. Gellman, N. Easton, MA (US); Michael Madden, Princeton, MA (US)

Correspondence Address: COOLEY GODWARD LLP, ATTN: PATENT GROUP, 11951 FREEDOM DRIVE, SUITE 1700, ONE FREEDOM SQUARE- RESTON TOWN CENTER, RESTON, VA, 20190-5061, US

(73) Assignee: SciMed Life Systems, Inc., Maple Grove, MN

(21) Appl. No.: 10/388,284

(22) Filed: Mar. 13, 2003

(51) Int. Cl. A61F 2/04

(52) U.S. Cl. 604/8; 623/23.66; 623/23.71

(57) ABSTRACT

A stent with rapid return and strong shape memory prevents migration of the stent out of a desired site within a patient's body due to peristaltic actions. One particular stent includes an elongated member which defines an eccentric lumen extending longitudinally therethrough such that a first wall portion of the elongated member is thicker than a second wall portion of the elongated member. The first wall has a stronger shape memory than the second wall, and it recoils faster than the second wall. Another particular stent includes a co-extruded elongated member. The elongated member comprises a first material co-extruded with a second material where the second material forms at least a portion of a wall of the elongated member. The second material can have a higher modulus of elasticity than the first material. Additionally or alternatively, the second material can include a shape memory material.

### TECHNICAL FIELD

The invention relates generally to medical devices and, more particularly, to drainage stent with rapid return and strong shape memory.

### BACKGROUND INFORMATION

Medical devices are sometimes used to drain fluids from body cavities. For example, when a duct is blocked due to formation of stones, cysts, tumors or other causes of constriction, medical devices (such as drainage stents) are used to assist drainage of fluids by providing a conduit through the occluded duct. Stents can be used to drain fluids from the kidney to the bladder through the ureter, from the liver through the biliary ducts, from the gall bladder through the cystic, hepatic, or common bile ducts, and from the dorsal or ventral pancreas through the pancreatic ducts, for example.

One common design of a stent has a coil or pigtail on each end of the stent. These ends serve to anchor the tubular body of the stent in place within the patient's body. During placement, the stent typically is first straightened with a stylet inserted into the lumen of the stent, and then the straightened stent is inserted into the patient's body and placed in a duct, such as the ureter. The stylet is then removed, and the straightened coils automatically recoil and return to their coiled positions to anchor the stent in the patient. The time for the straightened coils to return to their coiled positions can be as long as thirty minutes. During this extended recoil time, peristaltic actions can cause the stent to migrate out of the duct. Further, even after the coils have reformed within the patient's body, the stent can be forced out of the duct due to stress exerted on the stent by the patient's normal bodily functions.

### SUMMARY OF THE INVENTION

The invention generally relates to drainage stents and other medical devices with rapid return and strong shape memory. In the present invention, a retaining shape located at least at one end of a medical device (such as a ureteral stent) is configured such that, after insertion into the patient, the retaining shape reforms more rapidly and has a stronger shape memory than known devices.

In one aspect, the invention involves a medical device comprising an elongated member for placement in a patient. The elongated member defines an eccentric lumen which extends longitudinally through the elongated member such that a first wall portion of the elongated member is thicker than a second wall portion of the elongated member. The elongated member includes a substantially straight portion adapted for placement in a duct and a proximal portion extending integrally from a proximal end of the substantially straight portion. The proximal portion is adapted to form a first retaining shape when placed within the patient's body to retain the substantially straight portion in the duct. The elongated member further includes a distal portion extending integrally from a distal end of the substantially straight portion. The distal portion is adapted to form a second retaining shape when placed within the patient's body to retain the substantially straight portion in the duct.

Embodiments according to this aspect of the invention can include various features. For example, the first and second retaining shapes can comprise coils. The first wall portion can be disposed along an inside or outside circumference of the coils. The elongated member can comprise a radiopaque material. The first wall portion can comprise a stronger shape memory (such as a stronger recoil memory) than the second wall portion. The first wall portion reforms (or recoils) faster than the second wall portion.

In another aspect, the invention involves a medical device comprising a coextruded elongated member for placement in a patient. The elongated member defines a lumen which extends longitudinally through the elongated member and includes a first material coextruded with a second material. The second material forms at least a portion of a wall of the elongated member. The elongated member includes a substantially straight portion adapted for placement in a duct and a proximal portion extending integrally from a proximal end of the substantially straight portion. The proximal portion is adapted to form a retaining shape when placed within the patient's body to retain the substantially straight portion in the duct. The proximal portion comprises the first and second materials.

Embodiments according to this aspect of the invention can include various features. For example, the elongated member may further include a distal portion extending integrally from a distal end of the substantially straight portion. The distal portion may be adapted to form a second retaining shape when placed within the patient's body to retain the substantially straight portion in the duct. The distal portion may include the first and second materials. The second material can extend between an inner and an outer surface of the wall of the elongated member. The second material also can extend longitudinally along at least the proximal and distal portions of the elongated member. The second material also can extend longitudinally along the substantially straight portion. The second material can be more rigid than the first material and can have a higher modulus of elasticity than the first material. The first material and the second material can melt at substantially the same temperature. The first and second retaining shapes can each include a coil. The second material can be disposed along an inside or an outside circumference of the coils. The second material can comprise a radiopaque material. The first material can include a first color and the second material can include a second color. The second material can form at least a portion of an outer surface of the wall of the elongated member. The second material can be a shape memory material. The shape memory material can have a stronger shape memory than the first material. The shape memory material can recoil faster than the first material.

The foregoing and other objects, aspects, features, and advantages of the invention will become more apparent from the following description and from the claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1A is an illustrative diagram of a drainage stent according to one embodiment of the invention.

FIG. 1B is a cross sectional view of the stent of FIG. 1A, taken along line B-B of FIG. 1A.

FIG. 1C is an illustrative diagram of a drainage stent with a retaining shape on only one and.

FIG. 1D is another illustrative diagram of a drainage stent with a retaining shape on only one end.

FIG. 1E is an illustrative diagram of a drainage stent with retaining shapes on both ends.

FIG. 1F is another illustrative diagram of a drainage stent with retaining shapes on both ends.

FIG. 1G is yet another illustrative diagram of a drainage stent with retaining shapes on both ends.

FIG. 1H is an illustrative diagram of the drainage stent shown in FIG. 1A straightened with a stylet.

FIG. 2 is an illustrative diagram of a drainage stent being inserted into a patient.

FIG. 3 is an illustrative diagram of the stent shown in FIG. 2 disposed within the patient's urinary system.

FIG. 4A is a perspective view of a portion of a stent including an eccentric lumen according to one embodiment of the invention.

FIG. 4B is a cross sectional view of the portion of the stent of FIG. 4A, taken along line B-B of FIG. 4A.

FIG. 5 is an illustrative view of a drainage stent with a second material disposed along an inside circumference of the retention coils according to one embodiment of the invention.

FIG. 6A is a perspective view of a portion of a stent including a first material coextruded with a second material, according to another embodiment of the invention.

FIG. 6B is a cross sectional view of the portion of the stent of FIG. 6A, taken along line B-B of FIG. 6A.

FIG. 6C is a perspective view of a portion of a stent including a first material coextruded with a second material, according to still another embodiment of the invention.

FIG. 6D is a cross sectional view of the portion of the stent of FIG. 6C, taken along line B-B of FIG. 6C.

FIG. 7 is an illustrative view of a drainage stent with a second material disposed along an outside circumference of the retention coils according to another embodiment of the invention.

FIG. 8A is an illustrative view of a drainage stent with a second material disposed along an inside circumference of the retention coils according to still another embodiment of the invention.

FIG. 8B is a perspective view of a portion of a stent including a first material co-extruded with a second material according to yet another embodiment of the invention.

FIG. 8C is a cross sectional view of the portion of the stent of FIG. 8B, taken along line B-B of FIG. 8B.

FIG. 8D is an illustrative view of a drainage stent with a second material disposed along an outside circumference of the retention coils according yet another embodiment of the invention.

### DESCRIPTION

The invention relates generally to medical devices, such as ureteral stents, with rapid return and strong shape memory features that are placed within a patient's body to permit fluid flow in a previously occluded body lumen. Drainage stents according to the invention generally include a retaining shape disposed on at least one end of a substantially straight portion. The retaining shape is configured to anchor the stent in a duct, such as the ureter, where a blockage has occurred. In the present invention, the retaining shapes, which can be, for example, loops, pigtails, or coils, and which can be different on either end if one shape is disposed on each end, are configured such that, after insertion into the patient, the retaining shapes reform more rapidly and have a stronger shape memory than the retaining shapes of existing drainage stents.

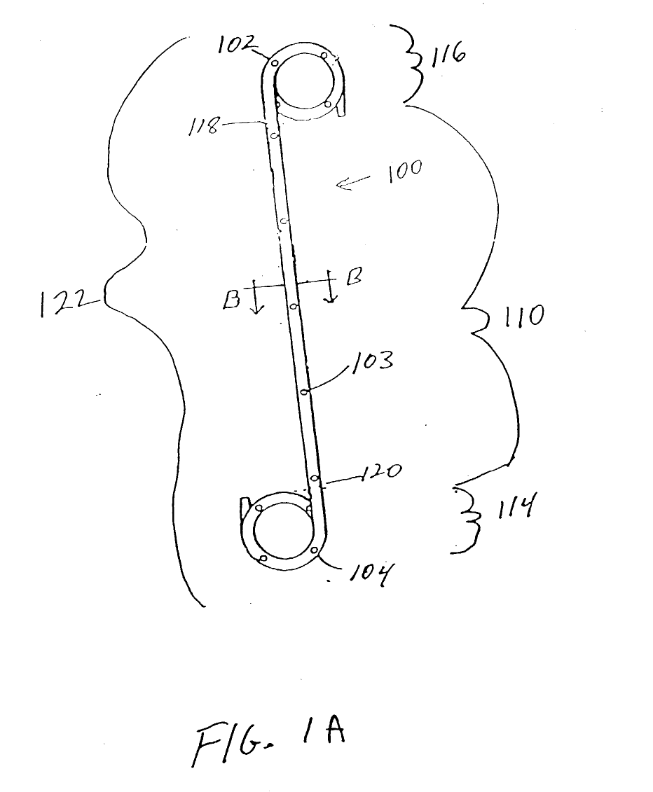

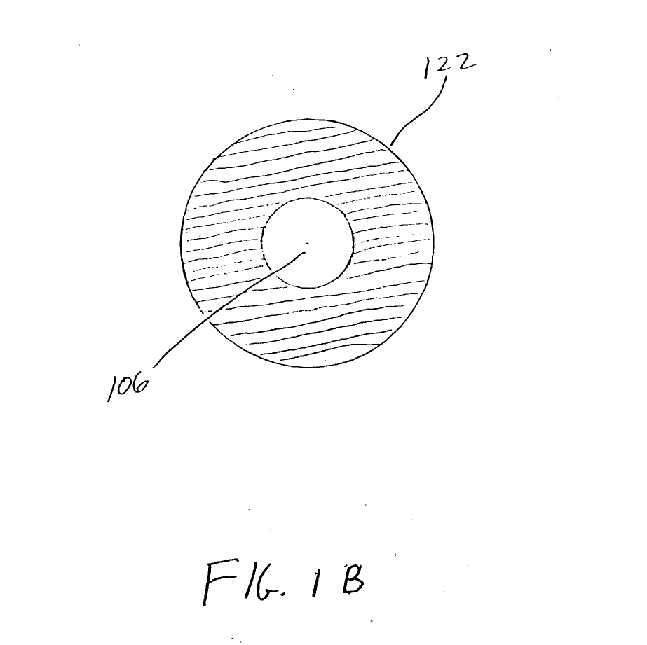

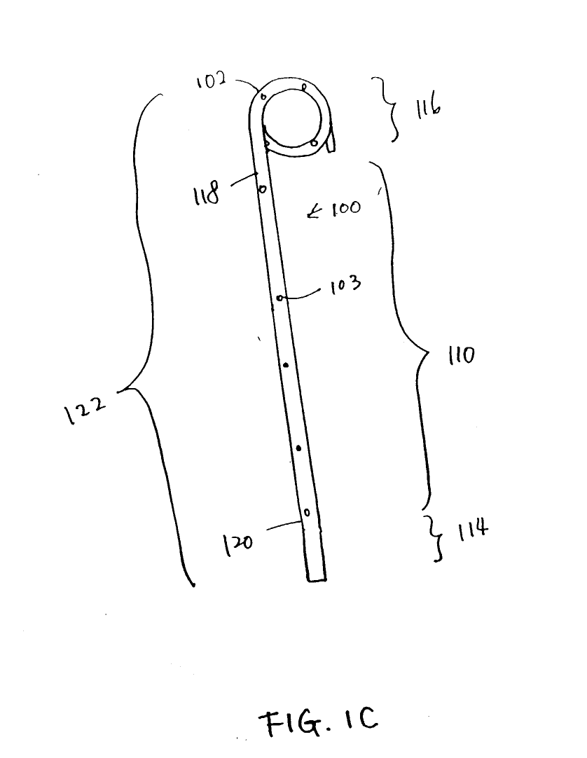

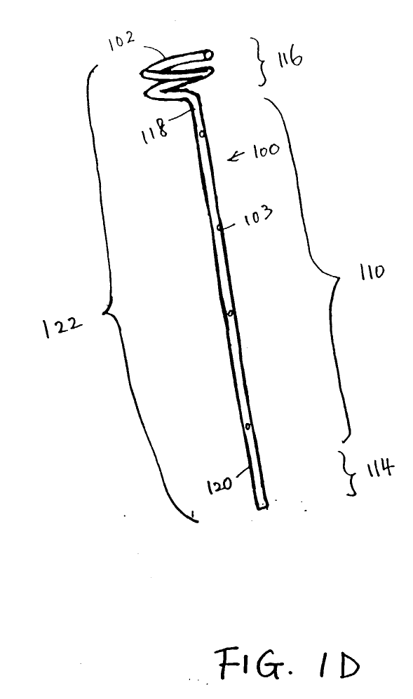

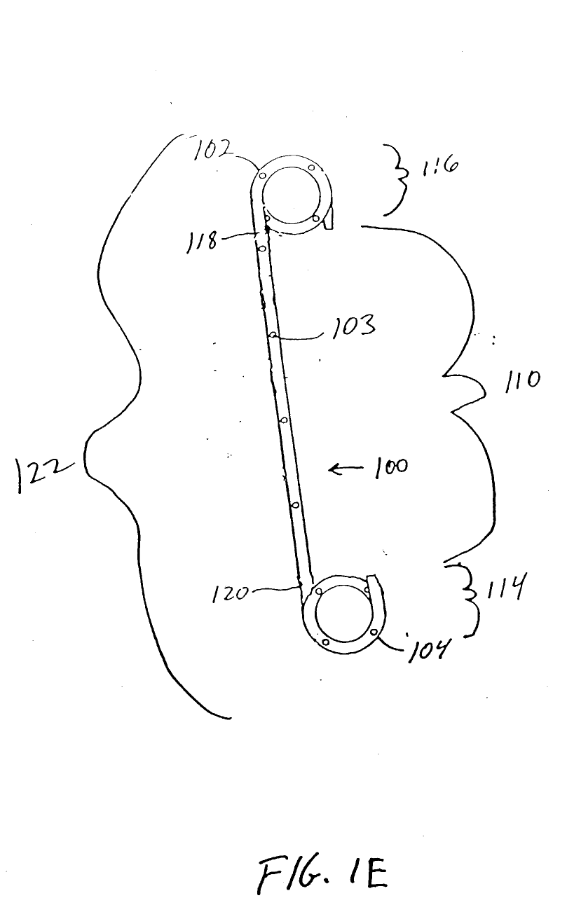

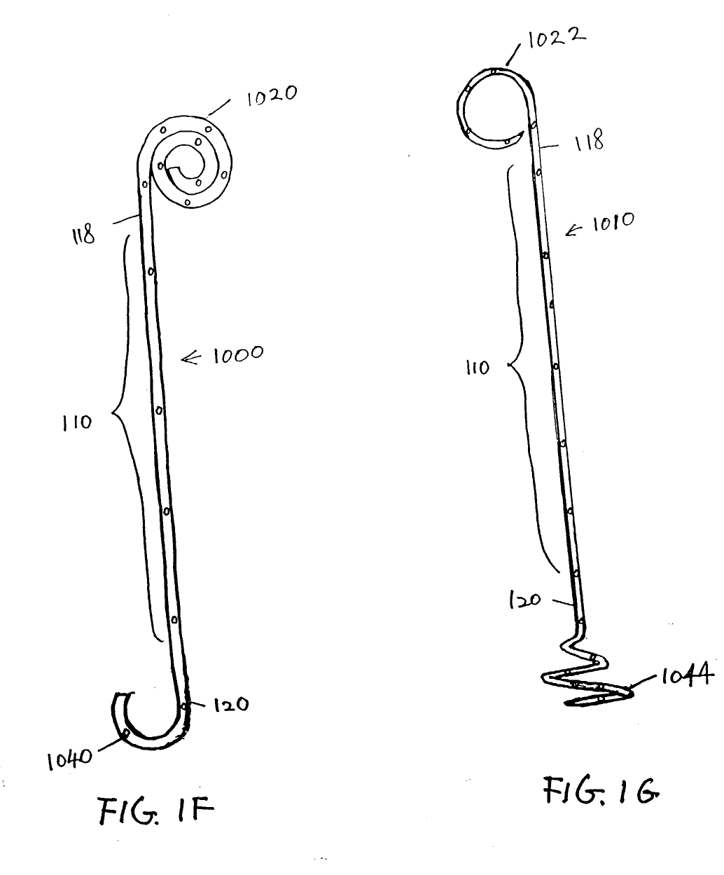

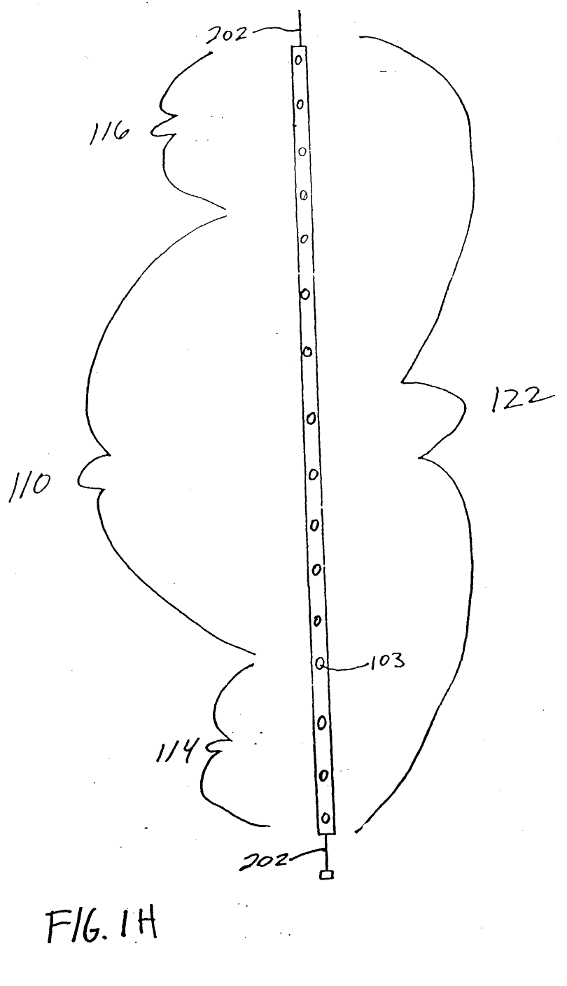

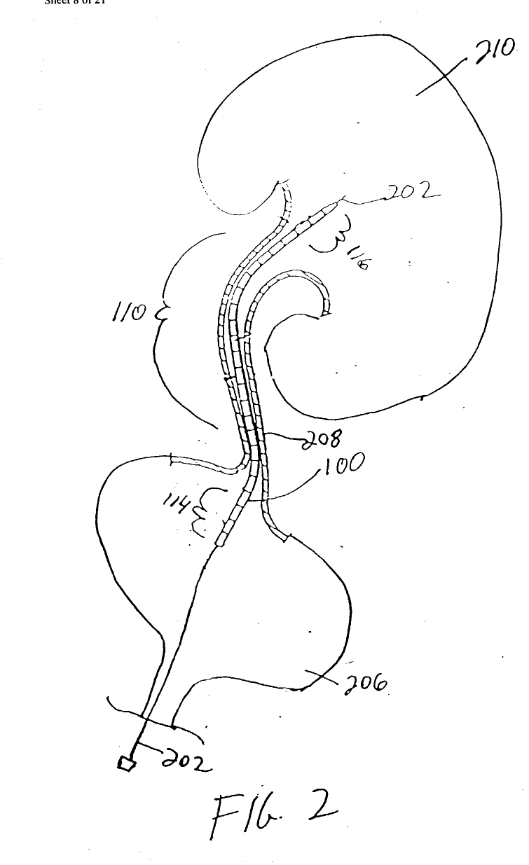

Referring to FIG. 1A, a drainage stent 100, such as a ureteral stent, includes a flexible elongated member 122 which defines a lumen 106 (shown in FIG. 1B) that extends longitudinally through the elongated member. The elongated member 122 includes a substantially straight portion 110, a proximal portion 114, and a distal portion 116. The proximal portion 114 extends integrally from a proximal end 120 of the substantially straight portion 110 and is adapted to form a first retaining shape (in, for example, the urinary bladder of the patient) to retain the substantially straight portion 110 in place in a duct (for example, the ureter). The distal portion 116 extends integrally from a distal end 118 of the substantially straight portion 110 and is adapted to form a second retaining shape (in, for example, a kidney of the patient) to retain the substantially straight portion 110 in place in the duct (for example, the ureter). In one embodiment, as shown in FIGS. 1C and 1D, the stent comprises a retaining shape only on the distal end 116. The retaining shape may be, for example, a coil that lies in substantially the same plane as the substantially straight portion, as shown in FIG. 1C. As another example, the retaining shape can be a coil that revolves about an axis that generally extends along the substantially straight portion, as shown in FIG. 1D. In another embodiment, as shown in FIGS. 1A and 1E, the stent comprises a first and second retaining shapes. In this embodiment, the retaining shapes are coils 102, 104 which extend integrally from, respectively, the distal and proximal ends 118, 120 of the substantially straight portion 110. In other words, the coils 102, 104 and the substantially straight portion 110 are not separate parts that are connected together to form the stent 100. Instead, the coils 102, 104 and the substantially straight portion 110 are sections of the single elongated member 122 which form the stent 100. The coils 102, 104, when in a retaining or coiled position, may coil in the opposite directions as shown in FIG. 1A, or coil in the same direction as shown in FIG. 1E. The coils 102, 104 can independently lie in substantially the same plane as the substantially straight portion 110, or they can revolve about an axis that generally extends along the substantially straight portion 110. In other embodiments, the retaining shapes that extend integrally from the distal and proximal ends 118, 120 of the substantially straight portion 110 can be, for example, loops, pigtails, hooks, or any combination thereof. The retaining shapes can lie in the same axial plane as one another, or they can lie in planes offset from one another. As shown in FIG. 1F, a ureteral stent 1000 can have retaining shapes 1020, 1040 in the shape of a multi-turn coil and a J-curl turning in opposite directions and lying within the same axial plane. Another ureteral stent 1010 can have retaining shapes 1022, 1044 in the shape of a hook and a helical coil lying on substantially perpendicular planes, as shown in FIG. 1G.

The elongated member 122 can be extruded from a variety of materials including, but not limited to, polyurethane, nylon, polypropylene, polyethylene, and blends thereof, silicone, ethylene vinyl acetate, fluorinated ethylene propylene, polytetrafluoroethylene, and thermoplastic rubber. In one embodiment of the invention, the elongated member 122 is extruded from nylon. The elongated member 122 may also include a plurality of drainage holes 103 disposed along the length of the elongated member 122 to allow fluids to easily drain into the lumen 106, and these holes 102 can be formed during or after extrusion.

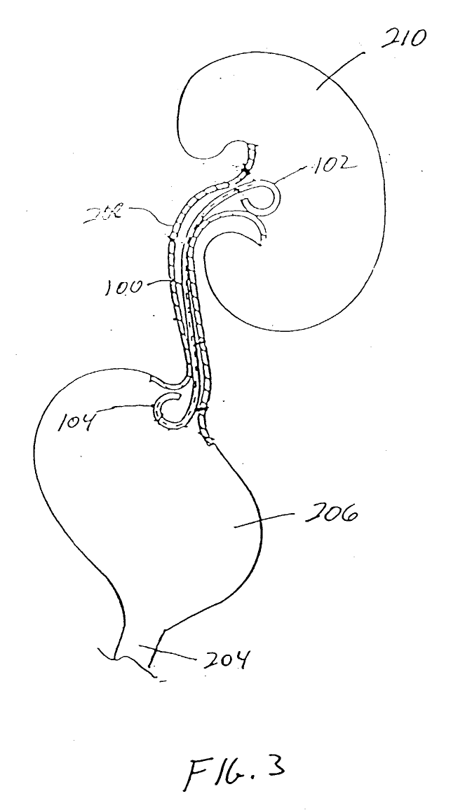

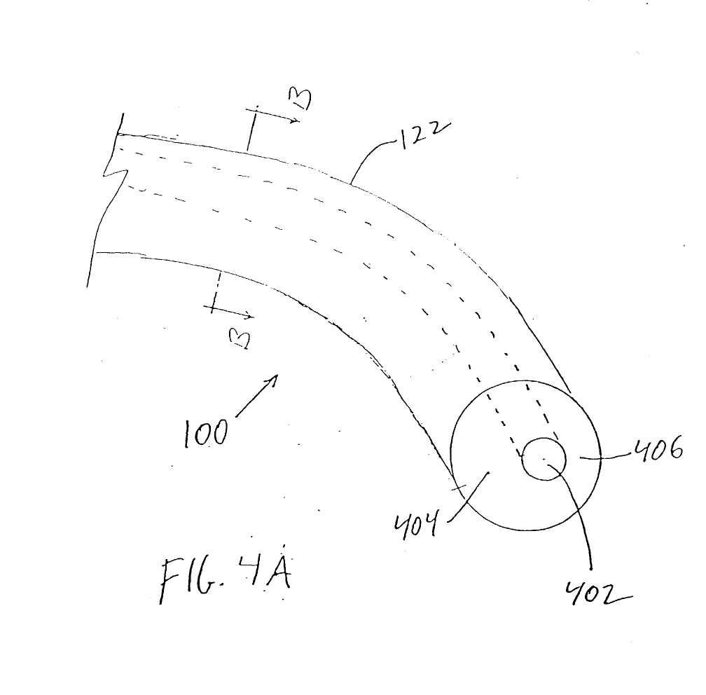

Referring to FIGS. 1H, 2, and 3, in operation, prior to the stent 100 being inserted into the patient (by a physician or other medical personnel), and for illustrative purpose more specifically into the patient's urinary system, the coils 102, 104 are straightened by inserting a stylet 202 through the lumen 106. The stent 100 is then inserted into the patient through the patent's urethra 204, guided through the patient's urinary bladder 206, and into the patient's ureter 208. The stent 100 is positioned in the patient's ureter 208 such that the distal portion 116 is disposed in the patient's kidney 210, the substantially straight portion 110 is disposed in the patient's ureter 208, and the proximal portion 114 is disposed in the patient's urinary bladder 206. After the stent 100 is positioned in the patient's urinary system, the stylet 202 is removed and the distal and proximal portions 116, 114 recoil to reform the coils 102, 104, respectively. The coils 102, 104 serve to hold the stent 100 in place in the patient's urinary system.

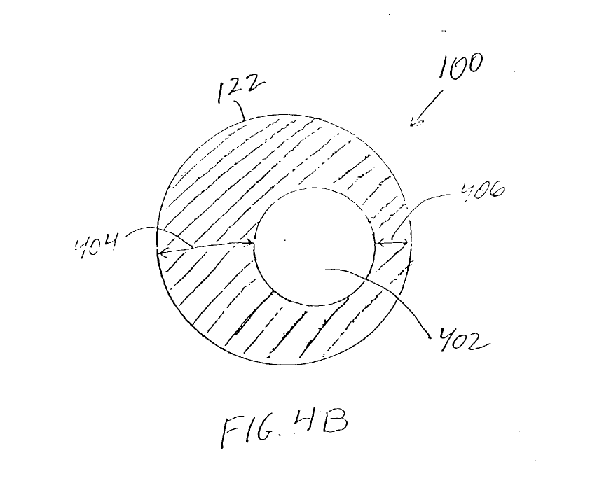

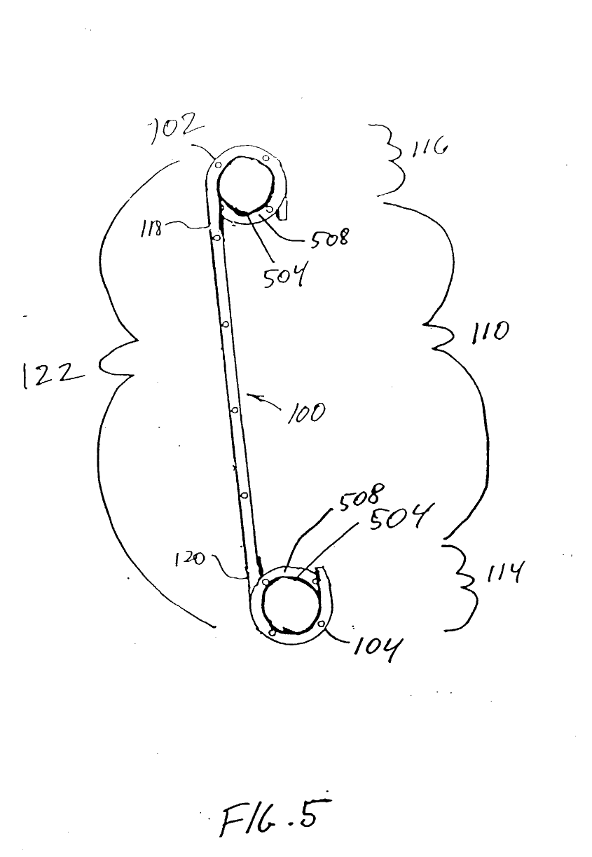

Referring to FIGS. 1A, 4A, and 4B, in one embodiment, the elongated member 122 of the stent 100 defines an eccentric lumen 402 that extends longitudinally through the elongated member such that a first wall 404 portion of the elongated member 122 is thicker than a second wall portion 406 of the elongated member 122. The first (thicker) wall portion 404 reforms or recoils (returns to a coiled position) faster than the second (thinner) wall portion 406. Further, the first wall portion 404 has a stronger shape memory than the second wall portion 406.

Shape memory is a feature of a retaining shape (such as a loop, pigtail, hook, or coil, for example) that allows the retaining shape to return to a particular shape after deformation and substantially retain it even when under tension from peristaltic actions. For example, a coil retaining shape with a strong shape memory will return to a coiled shape (after being straightened with a stylet and the stylet subsequently removed, for example) faster than a coil retaining shape with a weaker shape memory. Further, the coil retaining shape with the strong shape memory will be able to maintain the coiled shape when under tension (from peristaltic forces within the patient's body, for example) better than the coil retaining shape with the weaker shape memory.

In one embodiment, the first wall portion 404 is positioned on an inside circumference of the coils 102, 104 thereby pulling the distal portion 116 and the proximal portion 114 into the coiled position. In another embodiment, the first wall portion 404 is positioned on an outside circumference of the coils 102, 104 thereby pushing the distal portion 116 and the proximal portion 114 into the coiled position.

In some embodiments, the elongated member 122 includes a radiopaque material which facilitates easily locating the stent 100 (with, for example, a fluoroscope) when the stent 100 is disposed within the patient. The radiopaque material can be painted on, and/or embedded in, the elongated member 122. The painting typically is done after extrusion, whereas the embedding typically is done during extrusion of the elongated member 122. The radiopaque material can be mixed with or added to the extrusion material(s). The radiopaque material, however applied to and/or incorporated into the stent 100, can comprise barium sulfate, titanium oxide, or other heavy metals.

A benefit of this embodiment is that the stent 100 including the eccentric lumen 402 will reform the retaining coil (or other) shapes 102, 104 faster and maintain the retaining coil shapes 102, 104 better than typical concentric lumen stents thereby reducing the chances of the stent 100 uncoiling and migrating out of the desired site within the patient's body.

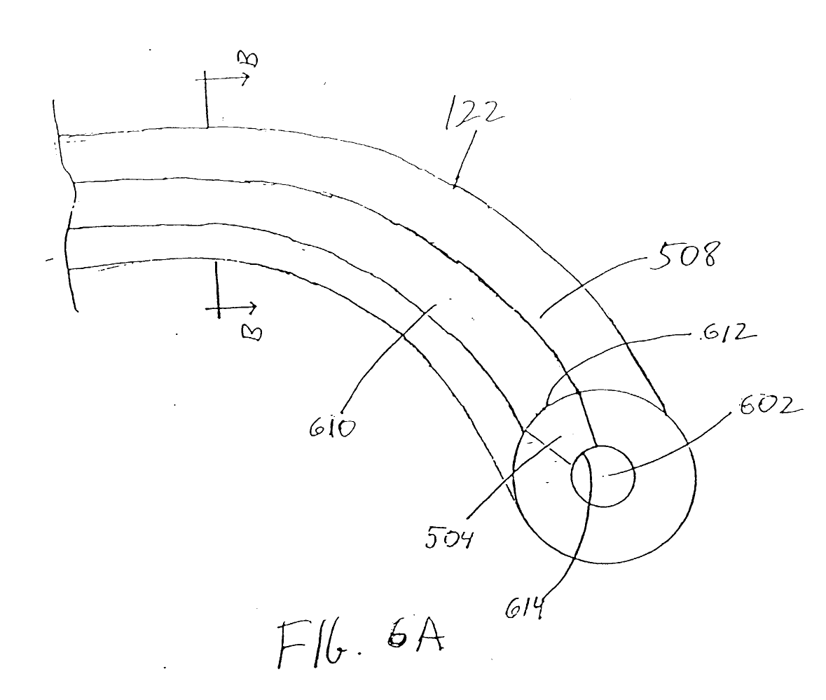

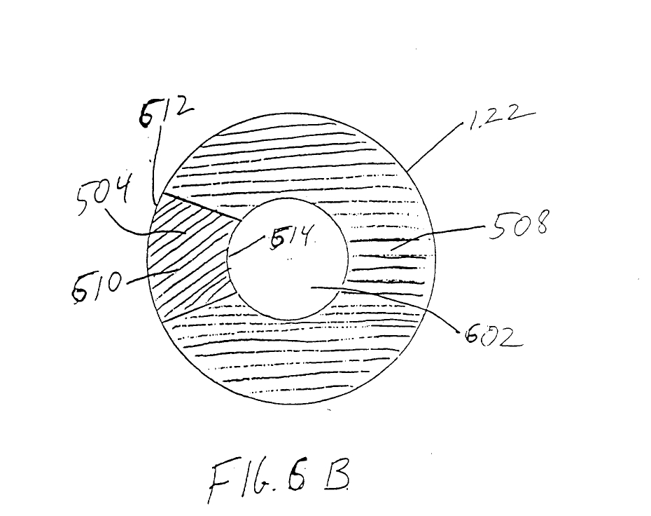

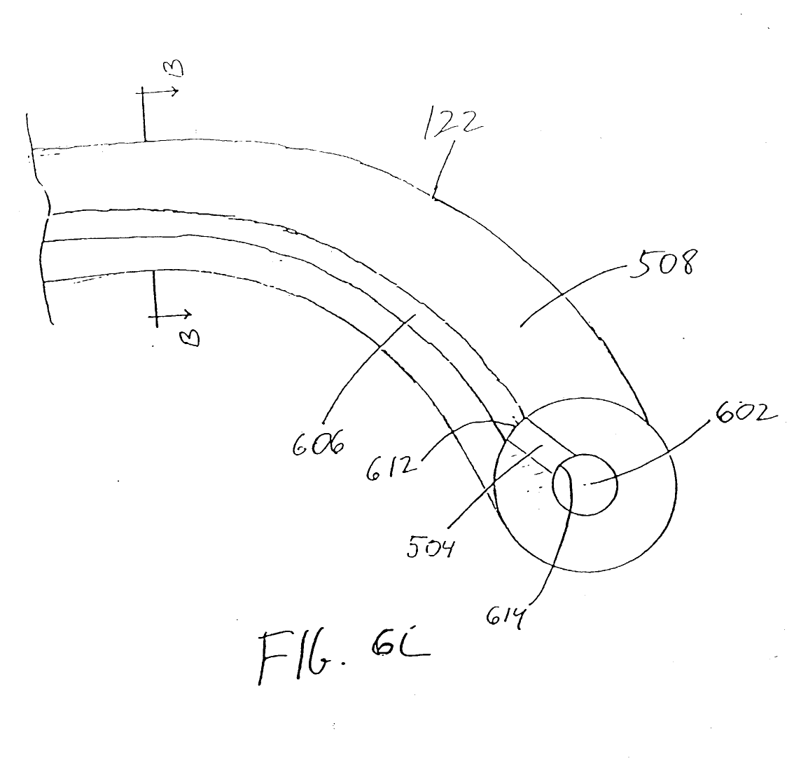

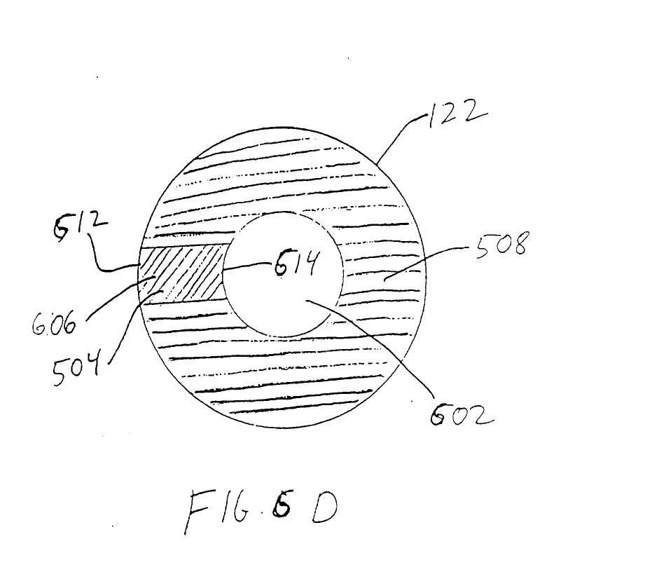

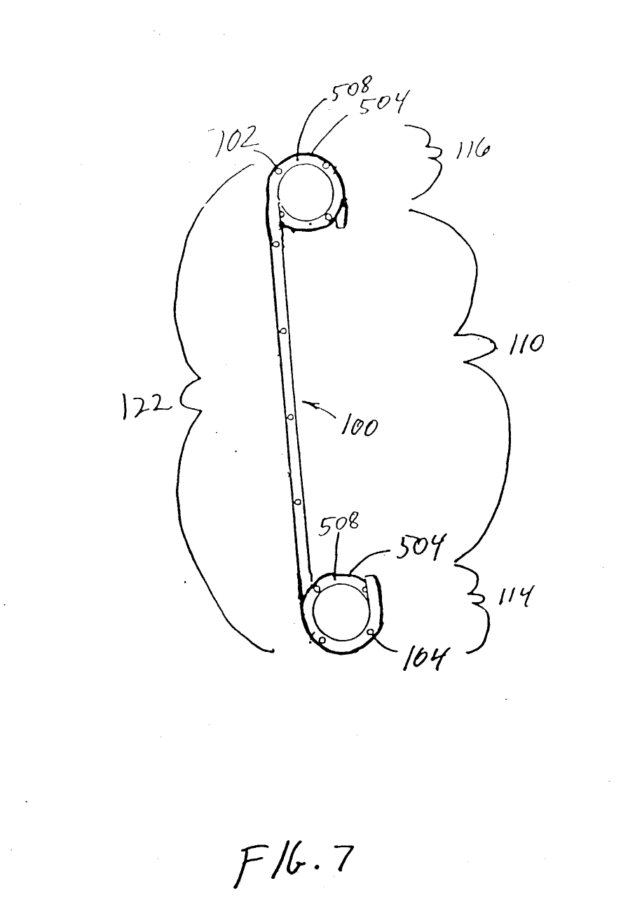

Referring to FIGS. 5, and 6A-6D, in another aspect, the elongated member 122 of the stent 100 comprises a first material 508 coextruded with a second material 504. The elongated member 122 defines a lumen 602 extending therethrough. The elongated member 122 includes a substantially straight portion 110 and a proximal portion 114. The proximal portion 114 extends integrally from a proximal end 120 of the substantially straight portion 110 and forms a first retaining shape 104 when placed in a first organ of the patient to retain the substantially straight portion 110 in place in a duct where a blockage has occured. In some embodiments, the proximal portion 114 of the elongated member 122 can be formed into a coil 104.

In one embodiment, the elongated member further includes a distal portion 116. The distal portion 116 extends integrally from a distal end 118 of the substantially straight portion 110 and forms a second retaining shape 102 when placed in a second organ of the patient to retain the substantially straight portion 110 in place in the duct. In some embodiments, the distal 116 and proximal 114 portions of the elongated member 122 are formed into coils 102, 104. In other embodiments, the distal 116 and proximal 114 portions of the elongated member 122 can be formed into other retaining shapes, such as loops, hooks, or pigtails, for example. In still other embodiments, the distal 116 and proximal 114 portions of the elongated member 122 can each be formed into different retaining shapes.

The second material 504 forms at least a portion of the wall of the elongated member 122. As shown in FIGS. 6A and 6C, the second material 504 can also form a portion of, and extend between, an inner surface 614 an outer surface 612 of the wall of the elongated member 122. The second material 504, which can be a wedge shape 610 (shown in FIGS. 6A and 6B) or a stripe shape 606 (shown in FIGS. 6C and 6D), also extends longitudinally along at least the proximal portion 114 and the distal portion 116 of the elongated member 122. In some embodiments, the second material 504 extends longitudinally along the entire length of the elongated member 122.

The first material 508 can be polyurethane, nylon, polypropylene, polyethylene, and blends thereof, silicone, ethylene vinyl acetate, fluorinated ethylene propylene, polytetrafluoroethylene, and thermoplastic rubber. In one embodiment of the invention, the first material is nylon. In another embodiment of the invention, the first material is polyurethane.

The second material 504 can be the same polymer as the first material 508, but of higher tensile strength. In the case where the second material 504 is a different polymer, the second material 504 is a shape memory polymer that has a melting temperature either identical to or substantially the same as the melting temperature of the first material 508. The melting temperatures of the first 508 and second 504 materials are substantially the same if the two materials can form distinct well-bonded sections within a single elongated member 122 when coextruded. The melting temperatures of the first 508 and second 504 materials are not substantially the same if the two materials delaminate during coextrusion or thereafter.

The second material 504 has a higher modulus of elasticity than the first material 508. A material's modulus of elasticity is a measure of the material's stiffness. Therefore, the first material 504 is more rigid than the second material 508. This characteristic enables the second material 504 to reform or recoil (return to a coiled position) faster than the first material 508. Further, the second material 504 has a stronger shape memory than the first material 508 which enables the second material 504 to reform and maintain the coiled retaining shape when under tension (from peristaltic forces exerted by the patient's urinary system, for example).

In some embodiments, the second material 504 includes a radiopaque material which facilitates easily locating the stent 100 (with, for example, a fluoroscope) when the stent 100 is disposed within the patient's body. The radiopaque material can be painted on, and/or embedded in, the elongated member 122. The radiopaque material can also be painted on, and/or embedded in, the first material 508 or the second material 504 alone. The painting typically is done after extrusion, whereas the embedding typically is done during extrusion of the elongated member 122. The radiopaque material can be mixed with or added to the extrusion material(s). The radiopaque material, however applied to and/or incorporated into the stent 100, can comprise barium sulfate, titanium oxide, or other heavy metals.

In still other embodiments, the first material 508 and the second material 504 are of different colors, thereby making the first material 508 distinguishable from the second material 504.

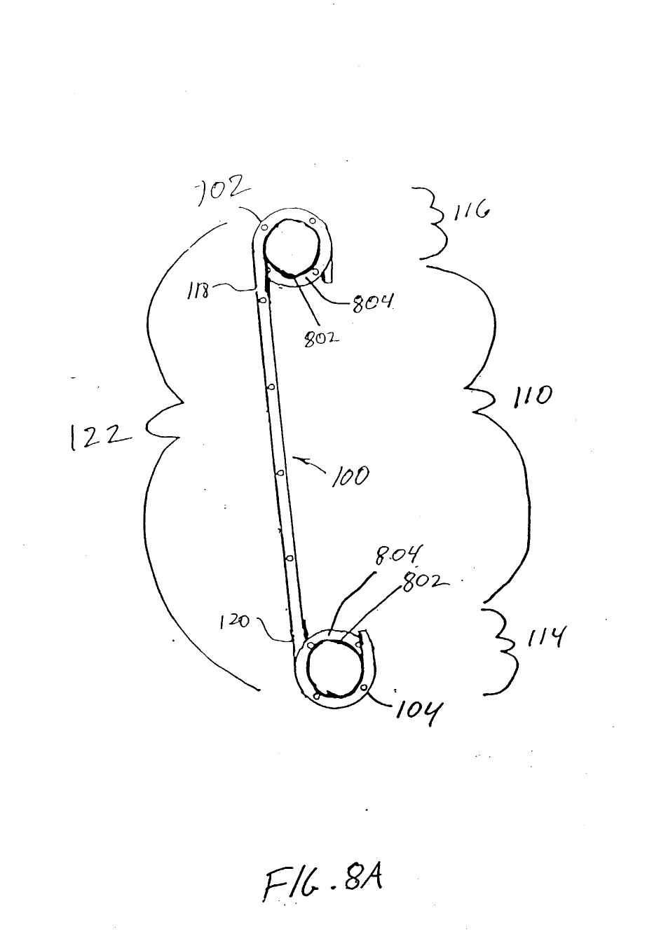

In one embodiment, the second material 504 is positioned on an inside circumference of the coils 102, 104 thereby pulling the distal portion 116 and the proximal portion 114 into the coiled position, as shown in FIG. 5. In another embodiment, the second material 504 is positioned on an outside circumference of the coils 102, 104 thereby pushing the distal portion 116 and the proximal portion 114 into the coiled position, as shown in FIG. 7.

A benefit of this embodiment is that the stent 100 including the first material 506 and the second material 504 will reform the retaining coil (or other) shapes 102, 104 faster and maintain the retaining coil shapes 102, 104 better than stents without the second material 504, thereby reducing the chances of the stent 100 uncoiling and migrating out of the desired site within the patient's body.

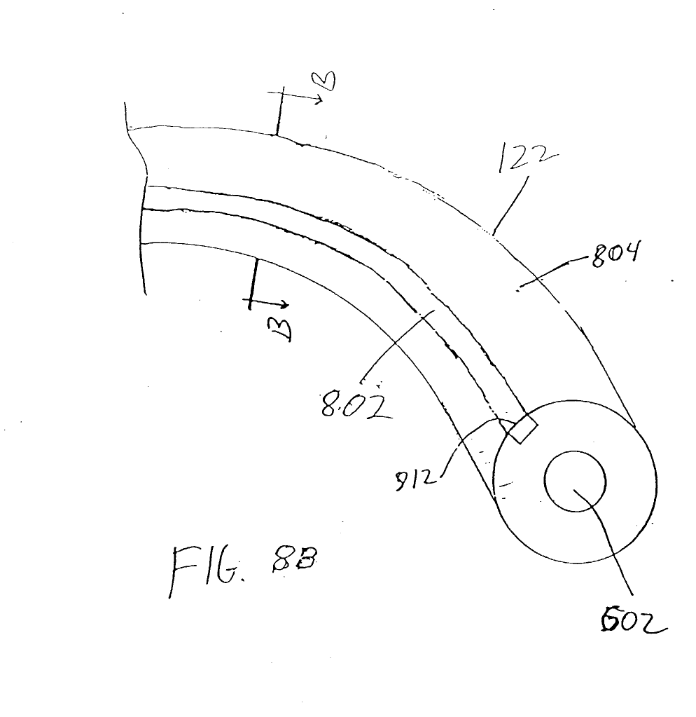

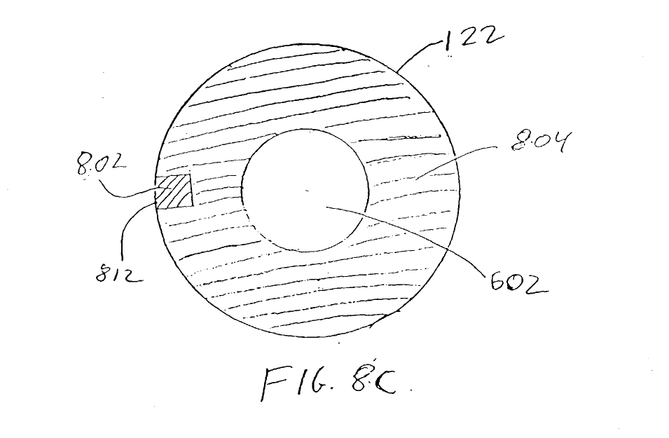

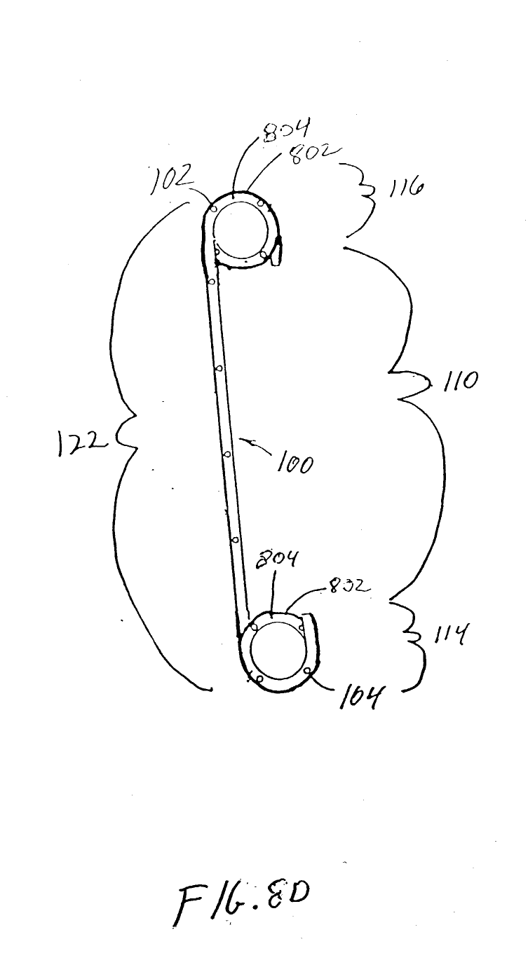

Referring to FIGS. 8A-8D, in another embodiment, the elongated member 122, which defines a lumen 602, includes a first material 804 co-extruded with a second material 802. The second material 802 forms a portion of an outer surface 812 of the wall of the elongated member 122 and also extends longitudinally along at least the proximal and distal portions 114, 116 of the elongated member 122. In some embodiments, the second material 802 extends longitudinally along the entire length of the elongated member 122.

The first material 804 can be polyurethane, nylon, polypropylene, polyethylene, and blends thereof, silicone, ethylene vinyl acetate, fluorinated ethylene propylene, polytetrafluoroethylene, and thermoplastic rubber. In one embodiment of the invention, the first material is polyurethane. In this embodiment, [0054]the second material 802 is a shape memory material. The second material 802 has a stronger shape memory than the first material 804 which enables the second material 802 to reform and maintain the coiled shape when under tension (from peristaltic forces exerted by the patient's urinary system, for example). An example of such a shape memory material that might be used as the second material 802 is Nitinol. Other shape memory alloys also can be used instead of or in conjunction with Nitinol.

In some embodiments, the second material 802 includes a radiopaque material which facilitates easily locating the stent 100 (with, for example, a fluoroscope) when the stent 100 is disposed within the patient's body. The radiopaque material can be painted on, or embedded in, the elongated member 122. The radiopaque material can also be painted on, or embedded in, the first material 508 or the second material 802 alone. The radiopaque material, however applied to and/or incorporated into the stent 100, can comprise barium sulfate, titanium oxide, or other heavy metals.

In still other embodiments, the first material 804 and the second material 802 are of different colors, thereby making the first material 804 distinguishable from the second material 802.

In one embodiment, the second material 802 is positioned on an inside circumference of the coils 102, 104 thereby pulling the distal portion 116 and the proximal portion 114 into the coiled position, as shown in FIG. 8A. In another embodiment, the second material 802 is positioned on an outside circumference of the coils 102, 104 thereby pushing the distal portion 116 and the proximal portion 114 into the coiled position, as shown in FIG. 8D.

A benefit of this embodiment is that the stent 100 including the second material 802 will reform the retaining coil (or other) shape 102, 104 faster and maintain the retaining coil shape 102, 104 better than stents without the second material 802, thereby reducing the chances of the stent 100 uncoiling and migrating out of the desired site within the patient's body.

Variations, modifications, and other implementations of what is described herein may occur to those of ordinary skill in the art without departing from the spirit and scope of the invention. Accordingly, the invention is not to be defined only by the preceding illustrative description.

What is claimed is:

1. A medical device, comprising:
an elongated member defining an eccentric lumen extending longitudinally therethrough such that a first wall portion of the elongated member is thicker than a second wall portion of the elongated member, the elongated member comprising:
a substantially straight portion adapted for placement in a duct within a patient's body;
a proximal portion extending integrally from a proximal end of the substantially straight portion, the proximal portion forming a first retaining shape when placed within the patient's body to retain the substantially straight portion in place in the duct; and
a distal portion extending integrally from a distal end of the substantially straight portion, the distal portion forming a second retaining shape when placed within the patient's body to retain the substantially straight portion in place in the duct.

2. The medical device of claim 1 wherein each of the first and second retaining shapes comprise a coil.

3. The medical device of claim 2 wherein the first wall portion is disposed along an inside circumference of each of the coils.

4. The medical device of the claim 2 wherein the first wall portion is disposed along an outside circumference of each of the coils.

5. The medical device of claim 1 wherein the elongated member comprises a radiopaque material.

6. The medical device of claim 2 wherein the first wall portion further comprises a stronger shape memory than the second wall portion.

7. The medical device of claim 2 wherein the first wall portion recoils faster than the second wall portion.

8. A medical device, comprising:
a co-extruded elongated member defining a lumen extending longitudinally therethrough, the elongated member comprising a first material co-extruded with a second material, the second material forming at least a portion of a wall of the elongated member, the elongated member comprising:
a substantially straight portion adapted for placement in a duct within a patient's body; and
a proximal portion extending integrally from a proximal end of the substantially straight portion, the proximal portion comprising the first and second materials and adapted to form a retaining shape when placed within the patient's body to retain the substantially straight portion in place in the duct.

9. The medical device of claim 8 wherein the elongated member further comprises a distal portion extending integrally from a distal end of the substantially straight portion, the distal portion comprising the first and second materials and adapted to form a second retaining shape when placed within the patient's body to retain the substantially straight portion in place in the duct.

10. The medical device of claim 9 wherein the second material extends between an inner surface of the wall and an outer surface of the wall.

11. The medical device of claim 10 wherein the second material also extends longitudinally in at least the proximal and distal portions of the elongated member.

12. The medical device of claim 11 wherein the second material also extends longitudinally in the substantially straight portion of the elongated member.

13. The medical device of claim 12 wherein the second material is more rigid than the first material.

14. The medical device of claim 8 wherein the second material has a higher modulus of elasticity than the first material.

15. The medical device of claim 8 wherein the first and second materials melt at substantially the same temperature.

16. The medical device of claim 9 wherein each of the retaining shapes comprises a coil.

17. The medical device of claim 16 wherein the second material is disposed along an inside circumference of each of the coils.

18. The medical device of claim 16 wherein the second material is disposed along an outside circumference of each of the coils.

19. The medical device of claim 8 wherein the second material comprises a radiopaque material.

20. The medical device of claim 8 wherein the first material includes a first color and the second material includes a second color.

21. The medical device of claim 9 wherein the second material forms at least a portion of an outer surface of the wall.

22. The medical device of claim 21 wherein the second material also extends longitudinally in at least the proximal and distal portions of the elongated member.

23. The medical device of claim 22 wherein the second material also extends longitudinally in the substantially straight portion of the elongated member.

24. The medical device of claim 8 wherein the second material comprises a shape memory material.

25. The medical device of claim 24 wherein the shape memory material comprises a stronger shape memory than the first material.

26. The medical device of claim 24 wherein the shape memory material recoils faster than the first material.
